(19) United States
(12) Patent Application Publication
(10) Pub. No.: US 2015/0025305 A1
(43) Pub. Date: Jan. 22, 2015

(54) SCROTAL CUSHION THERAPEUTIC DEVICE

(71) Applicants: Matthew Thompson STRINGER, (US); Emily Grace Reynolds STRINGER, (US)

(72) Inventors: Matthew Thompson Stringer, San Antonio, TX (US); Emily Grace Reynolds Stringer, San Antonio, TX (US)

(21) Appl. No.: 14/372,531

(22) PCT Filed: Jan. 18, 2013
(86) PCT No.: PCT/US2013/022055

§ 371 Date: Jul. 16, 2014

Related U.S. Application Data
(60) Provisional application No. 61/589,342, filed on Jan. 21, 2012.

(51) Int. Cl. A61F 7/08; A61F 6/02

(52) U.S. Cl. CPC A61F 7/08; A61F 6/02; A61F 2007/0048
USPC 600/33; 128/842; 607/108; 607/104

(57) ABSTRACT

A scrotal cushion and therapeutic device is provided that fits securely under and around the scrotum when a patient is seated or supine (body position characterized by lying flat on one's back). The scrotal cushion comprises a device body, a cap, four lateral wing elements contiguous with the scrotal cushion body, a temperature altering element (cooling or heating) and/or a receptacle for receiving the temperature altering element, and aeration elements. The body, cap and wings are preferably constructed of a soft, absorbent, antibacterial, antifungal, anti-decubitus, fast drying, durable, washable material. The scrotal cushion can be used to decrease pain, increase comfort, reduce scrotal swelling, improve hygiene, prevent scrotal skin tears, prevent infections, and alter fertility.

### CROSS-REFERENCE TO RELATED APPLICATIONS

This application claims priority to and the benefit of co-pending U.S. provisional patent application Ser. No. 61/589,342, entitled Scrotal Swelling Therapeutic Device, filed Jan. 21, 2012, which is incorporated herein by reference in its entirety.

### STATEMENT REGARDING FEDERALLY SPONSORED RESEARCH OR DEVELOPMENT

Not applicable

### 1. TECHNICAL FIELD

The present invention relates to scrotal support, cushion and therapy devices. The invention further relates to devices and methods for decreasing scrotal swelling and pain and for decreasing the morbidity associated with scrotal surgery or pathology. The invention also relates to devices and methods for increasing or decreasing fertility.

### 2. BACKGROUND OF THE INVENTION

Numerous conditions lead to a swollen or edematous scrotum including sharp, blunt or explosive trauma, infection, burns, hydrocele, inflammation, surgery, ascites (diffuse swelling throughout the body often due to liver failure) congestive heart failure, testicular torsion and inguinal hernia. A male soldier who is wounded by an improvised explosive device, for example, is likely to have a scrotal injury and limited mobility to perform his own wound care. In this scenario the scrotum can swell to 5-6 times its normal size. Other injuries aside, the scrotal swelling is painful, hampers the patient's mobility and impairs his ability to perform his activities of daily life. It also thins the scrotal skin putting the patient at risk for skin tears and ulceration.

The nursing care of patients with abdominal or groin wounds is complicated by excessive fluids that ooze from the wounds down into the groin. Stool and urine also tend to collect under the scrotum in bed-bound patients. The perineum (the diamond shaped area on the inferior surface of the trunk that includes the anus and scrotum) is the most dependent portion of the body when seated or supine (body position characterized by lying flat on one's back). Open wounds, stool and urine drain towards the perineum leading to a problematic accumulation of moisture. Keeping the scrotum and perineum clean and dry is very taxing on health care providers both in the institutional setting and at home. The retained moisture around the scrotum is uncomfortable for the patient and puts the patient at further risk for bacterial and fungal infections, superficial skin tears and decubitus ulcers.

Rest, ice, and scrotal elevation is the approach used by medical providers to decrease scrotal swelling and pain though there is little technology to assist with this technique. Positioning the scrotum on an ice pack that rests on a bundled towel is a common method of achieving the above. Unfortunately, this towel/ice technique has significant flaws. It traps moisture in the perineum and in the surrounding skin creases, the towel abrades the thinned scrotal skin, the ice pack easily falls off the towel and the towel and or ice must frequently be repositioned as the patient moves. Scrotal support surgical positioners are known in the art (see, e.g., US 20050085752 by Sells et al.) but do not provide therapeutic benefits of cooling or drying, they can trap moisture in the groin and they are easily displaced requiring frequent repositioning.

Citation or identification of any reference in Section 2, or in any other section of this application, shall not be considered an admission that such reference is available as prior art to the present invention.

### 3. SUMMARY OF THE INVENTION

A scrotal cushion is provided comprising:

a scrotal cushion body (also referred to herein as a device body) 100, wherein the scrotal cushion body has a longitudinal (proximal-distal) axis 110 and a lateral axis 120, and wherein the scrotal cushion body comprises:
a proximal side 210 and a distal side 220,
first and second opposite (lateral) sides 230, wherein the first and second opposite sides are positioned opposite with respect to the lateral axis 120,
a top side 240,
a bottom side 260 opposite the top side,
at least one soft, absorbent material for providing a supporting substrate; and
first and second lateral wing elements 300 secured to each of the first and second sides respectively of the scrotal cushion body.

In one embodiment, the scrotal cushion can comprise a temperature altering element 400. In another embodiment of the scrotal cushion, the temperature altering element is an insulated heating or a cooling element.

In another embodiment, the scrotal cushion can comprise a fertility altering element. In another embodiment, the scrotal cushion can comprise any suitable scrotal therapeutic element known in the art. In one embodiment, the scrotal cushion can comprise a plurality of such elements.

In another embodiment of the scrotal cushion, the temperature altering element is selected from the group consisting of a cool pack, a refrigeration device (e.g., a vapor-compression or absorption refrigeration device, a thermoelectric cooling (Peltier) device, a heat pack, a heating pad, and a thermoelectric heating device.

In another embodiment, the scrotal cushion can comprise a receptacle or tray 420 for receiving a temperature altering element, a fertility altering element or scrotal therapeutic element.

In another embodiment of the scrotal cushion, the receptacle for receiving the temperature altering element comprises a removable cap or lid 430.

In another embodiment of the scrotal cushion, the removable cap or lid 430 is positioned on the top side, the bottom side, the proximal side, the distal side or the first or second opposite side.

In another embodiment of the scrotal cushion, the first and the second lateral wing 300 elements are secured on each of the first and second sides.

In another embodiment of the scrotal cushion, the first or second lateral wing element 300 comprises an upper wing portion 301 and a lower wing portion 320.

In another embodiment of the scrotal cushion, at least one aeration element 270 is positioned in the scrotal cushion body. In a specific embodiment, the at least one aeration element is a port (270 in FIG. 1) or a tube (520 in FIG. 3). In another embodiment, the scrotal cushion can comprise both a port and a tube. In another embodiment, a second aeration element (e.g., an active air circulator or a fan) can be positioned within the port or tube.

In another embodiment the scrotal cushion or portions thereof can be impregnated with at least one bactericidal, bacteriostatic and/or anti-fungal substance. In a specific embodiment, the scrotal cushion body, lateral wings and/or cap are impregnated with the at least one bactericidal, bacteriostatic and/or anti-fungal substance.

A method is provided for altering the temperature of the scrotum of an individual. In one embodiment, the method comprises the steps of:

providing the scrotal cushion, wherein the temperature altering element is actively cooling or heating or the fertility altering element is active, or

providing the scrotal cushion, wherein the receptacle for receiving the temperature altering element contains a temperature altering element that is actively cooling or heating or a fertility altering element that is active:

elevating the individual's scrotum on the scrotal cushion, thereby positioning the scrotum to receive cooling or heating from the temperature altering element or activity from the fertility altering element;

inserting the first lateral wing element into the groin crease on a corresponding first side of the individual's body and under the leg on that side, thereby compressing the first lateral wing element;

inserting the second lateral wing element into the groin crease on the corresponding second side of the individual's body and under the leg on that side, thereby compressing the second wing element; and

securing the scrotal cushion under the scrotum,

thereby altering the temperature of the scrotum and/or altering the fertility of the individual.

The methods provided herein can be used with male mammals, including but not limited to human, cat, dog, cow, horse, pig, sheep, goat, exotic (e.g., zoo) mammal, etc.

A method is provided for inhibiting or promoting fertility in an individual. In one embodiment, the method comprises the steps of:

providing the scrotal cushion, wherein the temperature altering element is actively cooling or heating or the fertility altering element is active to inhibit or promote fertility, or

providing the scrotal cushion, wherein the receptacle for receiving the temperature altering element contains a temperature altering element that is actively cooling or heating or a fertility altering element that is active to inhibit or promote fertility;

elevating the individual's scrotum on the scrotal cushion, thereby positioning the scrotum to receive cooling or heating from the temperature altering element or activity from the fertility altering element;

inserting the first lateral wing element into the groin crease on a corresponding first side of the individual's body and under the leg on that side, thereby compressing the first lateral wing element;

inserting the second lateral wing element into the groin crease on the corresponding second side of the individual's body and under the leg on that side, thereby compressing the second wing element; and

securing the scrotal cushion under the scrotum,

thereby altering the temperature of the scrotum and/or altering the fertility of the individual.

A method is provided for promoting decreased sperm count or spermatogenesis in an individual. In one embodiment, the method comprises the steps of:

providing the scrotal cushion, wherein the temperature altering element is actively heating or the fertility altering element is active to decrease sperm count, or providing the scrotal cushion, wherein the receptacle for receiving the temperature altering element contains a temperature altering element that is actively heating or a fertility altering element that is active to decrease sperm count;

elevating the individual's scrotum on the scrotal cushion, thereby positioning the scrotum to receive heating from the temperature altering element or activity from the fertility altering element;

inserting the first lateral wing element into the groin crease on a corresponding first side of the individual's body and under the leg on that side, thereby compressing the first lateral wing element;

inserting the second lateral wing element into the groin crease on the corresponding second side of the individual's body and under the leg on that side, thereby compressing the second wing element; and

securing the scrotal cushion under the scrotum,

thereby raising the temperature of the scrotum and/or decreasing the fertility of the individual.

A method is provided for promoting increased sperm count or spermatogenesis in an individual. In one embodiment, the method comprises the steps of:

providing the scrotal cushion, wherein the temperature altering element is actively cooling or the fertility altering element is active to increase sperm count, or providing the scrotal cushion, wherein the receptacle for receiving the temperature altering element contains a temperature altering element that is actively cooling or a fertility altering element that is active to increase sperm count;

elevating the individual's scrotum on the scrotal cushion, thereby positioning the scrotum to receive cooling from the temperature altering element or activity from the fertility altering element;

inserting the first lateral wing element into the groin crease on a corresponding first side of the individual's body and under the leg on that side, thereby compressing the first lateral wing element;

inserting the second lateral wing element into the groin crease on the corresponding second side of the individual's body and under the leg on that side, thereby compressing the second wing element; and

securing the scrotal cushion under the scrotum,

thereby lowering the temperature of the scrotum and/or increasing the fertility of the individual.

A method is provided for positioning the scrotum of an individual. In one embodiment, the method comprises the steps of:

providing the scrotal cushion;

elevating the individual's scrotum on the scrotal cushion, thereby positioning the scrotum on the scrotal cushion;

inserting the first lateral wing element into the groin crease on a corresponding first side of the individual's body and under the leg on that side, thereby compressing the first lateral wing element;

inserting the second lateral wing element into the groin crease on the corresponding second side of the individual's body and under the leg on that side, thereby compressing the second wing element; and

securing the scrotal cushion under the scrotum.

The method decreases the adjustments needed to secure the scrotal cushion in place in a supine or seated patient.

A method is provided for altering fertility or semen parameters in an individual. The fertility or semen parameters can be measured, for example, by semen analysis. The method for altering fertility or semen parameters can comprise the steps of:

providing a scrotal cushion as disclosed herein, wherein the temperature altering element is actively cooling or heating, the fertility altering element is active, the receptacle for receiving the temperature altering and/or fertility altering element contains a temperature altering element that is actively cooling or heating and/or a fertility altering element that is active;

elevating the individual's scrotum on the scrotal cushion, thereby positioning the scrotum to receive cooling or heating from the temperature altering element or activity from the fertility altering element;

inserting wing elements of the scrotal cushion into groin creases on each side of the individual's body and under the leg on that side, thereby compressing the wing element; and

securing the scrotal cushion under the scrotum,

thereby altering the temperature of the scrotal contents and/or increasing or decreasing fertility.

### 4. BRIEF DESCRIPTION OF THE DRAWINGS

The present invention is described herein with reference to the accompanying drawings, in which similar reference characters denote similar elements throughout the several views. It is to be understood that in some instances, various aspects of the invention may be shown exaggerated or enlarged to facilitate an understanding of the invention. Broken line portions of the figure drawings are included to show unclaimed subject matter only and form no part of the claimed design.

FIG. 1 is a perspective view of one embodiment of the scrotal cushion 100.

FIG. 2 is another perspective view of one embodiment of the scrotal cushion 100, showing a cover, cap or lid 430 to be inserted over a temperature altering (cooling or heating) element 400 in the scrotal cushion. Platform, tray or receptacle for the temperature altering (cooling or heating) element 420.

FIG. 3 is another perspective view of one embodiment of the scrotal cushion 100, showing the cover, cap or lid 430 to be inserted over a temperature altering (cooling or heating) element 400 in the scrotal cushion. Platform, tray or receptacle for the temperature altering (cooling or heating) element 420. In this embodiment, the platform, tray or receptacle 420 is removable.

FIG. 4 shows an embodiment of the scrotal cushion 100 in use and positioned to support the scrotum.

### 5. DETAILED DESCRIPTION OF THE INVENTION

A scrotal support and therapy device (also referred to herein as “scrotal cushion”) for simultaneously elevating, drying and altering the temperature of the scrotum is provided. The scrotal cushion significantly improves the quality of care for the swollen tender scrotum. The scrotal cushion also is an effective therapeutic device for altering fertility. The scrotal cushion is suited for use in the seated or supine patient.

A commonly used technique for treating scrotal swelling in a supine or seated patient is to elevate the scrotum with a rolled-up towel, with or without an ice pack positioned under the scrotum. The towel used for elevation can trap unwanted moisture in the groin, does not reach into the creases lateral to the scrotum, may abrade the skin of the scrotum, and the ice pack often falls off. The scrotal cushion disclosed herein overcomes the aforementioned shortcomings by simultaneously elevating, drying, cooling and protecting the swollen tender scrotum. A scrotal support and therapy device for altering fertility by way of either cooling or heating the scrotum is also provided.

For clarity of disclosure, and not by way of limitation, the detailed description of the invention is divided into the subsections set forth below.

### 5.1. Scrotal Cushion Body

A scrotal cushion is provided that fits securely under and around the scrotum when the patient is seated or supine.

In one embodiment, the scrotal cushion comprises:

a scrotal cushion body 100 (also referred to herein as a device body), wherein the scrotal cushion body has a longitudinal (proximal-distal) axis 110 and a lateral axis 120, and wherein the device body comprises:
a proximal side 210 and a distal side 220,
first and second opposite sides 230, wherein the first and second opposite sides are positioned opposite with respect to the lateral axis 120,
a top side 240,
a bottom side 260 opposite the top side,
at least one soft, absorbent material for providing a supporting substrate; and
first and second lateral wing elements 300 secured to each of the first and second sides respectively of the device body.

In a preferred embodiment, the scrotal cushion comprises a temperature altering element or fertility altering element 400.

In one embodiment, the temperature altering element is a heating or a cooling element.

In another embodiment, the scrotal cushion comprises a receptacle for receiving a temperature altering element 420.

In another embodiment, the scrotal cushion comprises both a temperature altering (cooling or heating) element 400 and a receptacle for receiving a temperature altering (cooling or heating) element 420.

In another embodiment a fan or air circulating element is secured within the aeration tubes to actively circulate air under and around the swollen tender scrotum thus keeping the scrotum dry and comfortable.

The scrotal cushion body 100 is preferably constructed of at least one soft, absorbent material, such as super-absorbent foam or another wicking material known in the art, which is capable of wicking moisture off of the skin. In a preferred embodiment, the scrotal cushion body wicks moisture away from the perineum. The soft antidecubitus nature of the absorbent material serves to protect the thin scrotal skin. In one specific embodiment, a super absorbent material can be used such as hydrocolloid or absorbent foam.

In other embodiments, the lateral wing elements 300 and/or other elements of the scrotal cushion can comprise a material with the same beneficial properties as the body 100.

In one embodiment, the scrotal cushion is disposable and is to be replaced when soiled. In another embodiment it can be cleaned and re-used when soiled. It can be constructed of a soft malleable material to preserve the skin integrity and promote scrotal comfort. The malleable but supportive composition (cellulose, foam, hydrocolloid, gelatin or any other super absorbent material) will wick moisture out of the groin/perineum.

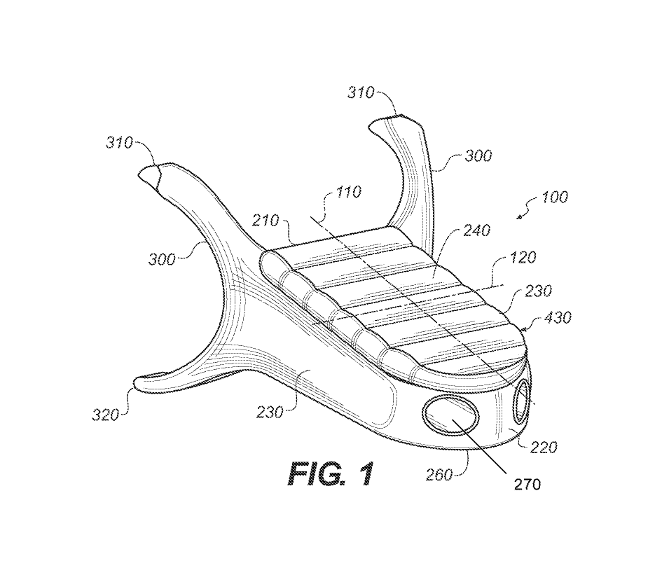

FIGS. 1-4 show various views of one embodiment of the scrotal cushion.

FIG. 1 is a perspective view of one embodiment of the scrotal cushion 100.

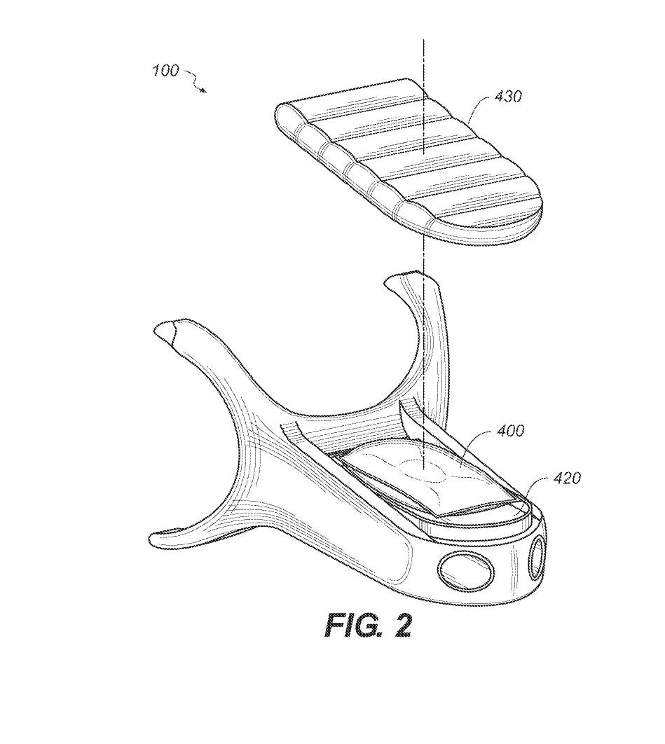

FIG. 2 is another perspective view of one embodiment of the scrotal cushion 100, showing a cover, cap or lid 430 to be inserted over a temperature altering (cooling or heating) element 400 in the scrotal cushion. Platform, tray or receptacle 420 for holding or receiving the temperature altering (cooling or heating) element.

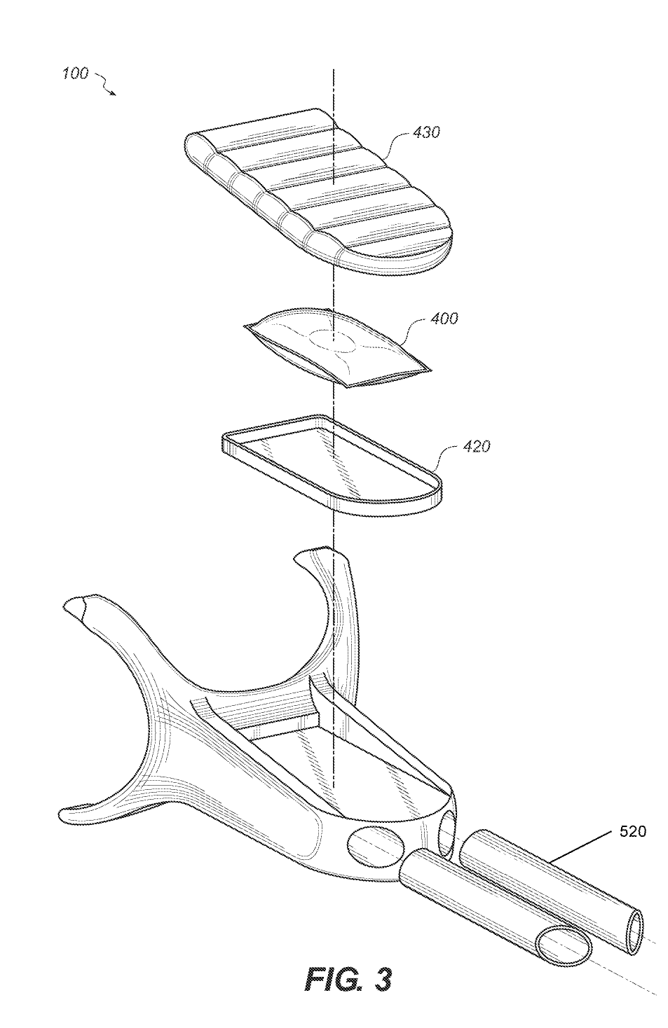

FIG. 3 is another perspective view of one embodiment of the scrotal cushion 100, showing the cover, cap or lid 430 to be inserted over a temperature altering (cooling or heating) element 400 in the scrotal cushion. Platform or receptacle 420 for the temperature altering (cooling or heating) element. In the embodiment depicted in FIG. 3, the receptacle 420 is removable to facilitate cleaning and draining of condensed moisture from the cooling element. In other embodiments, the scrotal cushion can lack the heating/cooling element or receptacle, and be used for support of the scrotum without cooling or heating the scrotum.

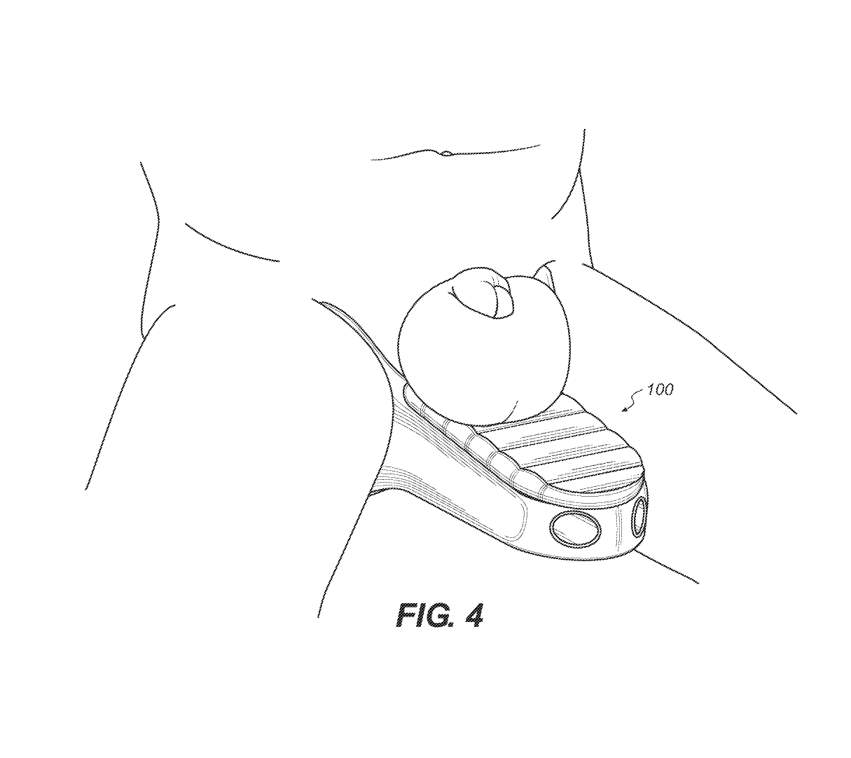

FIG. 4 shows an embodiment of the scrotal cushion 100 in use and positioned to support the swollen scrotum.

In one embodiment, the scrotal cushion or portions thereof can be impregnated with an antimicrobial, antifungal, bactericidal or bacteriostatic substance such as, but not limited to, silver. Impregnation with these substances can be used to prevent and/or treat soft tissue infections.

### 5.2. Wing Elements

The scrotal cushion body can comprise at least one lateral wing element 300. In one embodiment, a wing element can be secured to each of the first and second sides of the scrotal cushion body.

In a preferred embodiment, the scrotal cushion body comprises two wing elements, i.e., a left wing element and a right wing element secured to each side of the scrotal cushion body. In one embodiment, the wing element can comprise an upper wing portion 310 and a lower wing portion 320 (FIG. 1).

The scrotal cushion can be scaled in different sizes to be manufactured in adult, bariatric and pediatric sizes. The dimensions of the scrotal cushion can be scaled or custom-fitted by a skilled artisan to the measurements and requirements of a patient. For example, the sizes of juvenile, small adult and large adult versions can be scaled and adjusted accordingly. In the embodiment illustrated in FIG. 1, an adult-sized specific embodiment of the scrotal cushion can have a length of about 12 cm along central, proximal-distal axis 110 from the proximal side 210 to the distal side 220, about 8.5 cm between the opposite sides 230, about 15 cm between the lateral edges of the upper wing 310 portions of the two lateral wing elements 300, about 8 cm from the top side 240 to the bottom side 260 at the proximal side 210, about 5 cm from the top side 240 to the bottom side 260 at the distal side 220, and about a 20 cm diameter semi-circular opening across each wing, i.e., from the tip of the upper wing portion that inserts over the upper groin to the tip of the lower wing portion that inserts under the lower groin. The oval-shaped aeration elements (e.g., ports) 270 shown in FIG. 1 can have about a 3 cm diameter along their longest dimension. The art is not limited by the ratios implied by the sizes in the embodiment described above.

The lateral wing element(s) 300 fit into, and wick moisture out of, skin folds and/or crevices that are difficult to clean and keep dry. The lateral wing elements support the scrotum and keep the intertriginous areas of the groin/perineum dry. The lateral wing elements can be inserted into the groin creases on one side of the body and under the leg on that side. This keeps the scrotal cushion in place, minimizing the need for repositioning. When the upper wing portion 310 and lower wing portion 320 of the lateral wing elements 300 contact the skin in the crevices surrounding the scrotum, the skin in the crevices is kept dry by the wicking action of the material and the patient's skin compress the lateral wing elements, keeping the scrotal cushion secure under the scrotum.

In the embodiment of the scrotal cushion shown in FIGS. 1-4, the upper wing portion 310 of each wing element inserts over a leg of the patient. The lower wing portion 320 of each wing element inserts under a leg of the patient and is compressed by the patient's own weight, further securing the scrotal cushion in place underneath the scrotum.

### 5.3. Aeration Element

The scrotal cushion can comprise one or more aeration elements 270 in the scrotal cushion body for circulating fresh air under and around the swollen scrotum for comfort and drying. Aeration elements can be (or can comprise), for example, cylindrical tubes 520, as illustrated in FIG. 3 that can be inserted, fixed or fitted into an aeration port 270 (FIG. 1) on the scrotal cushion body. In another embodiment, the aeration element can comprise both a port and a tube that is an integral part of, or molded into, the scrotal cushion body. Aeration elements can be made of any suitable material known in the art, such as plastic, polymer or foam, and can be flexible or rigid. In a preferred embodiment, the aeration elements are tubes comprising (or composed of) a rigid plastic. In certain embodiments, aeration elements can be perforated with a plurality of holes to promote aeration. An aeration element can also comprise an active aeration device such as a fan. For example, a fan powered by alternating current, direct current or any other power source, could be implanted in the aeration tube or within the scrotal cushion body 100 to circulate air through the aeration tubes 520.

In a specific embodiment, the scrotal cushion comprises a plurality of hollow tubes 520 (FIG. 3) that serve as passive aeration elements and run through the base of the scrotal cushion body, e.g., into the aeration ports 270. The tubes allow for air exchange between the scrotal region and the surrounding air. The tubes can be, in certain embodiments, stiff (e.g., comprising a rigid plastic), so that they add to the structural integrity of the scrotal cushion. The air exchange provided by the aeration elements provides comfort to the patient and hastens drying of the scrotal/perineal region.

### 5.4. Temperature Altering and Fertility Altering Elements and Receptacles

In one embodiment, the scrotal cushion can comprise a temperature altering (cooling or heating) element 400 and/or a receptacle 420 for receiving a temperature altering (cooling or heating) element positioned in the scrotal cushion body. In the descriptions herein any cooling element can be replaced or interchanged with a heating element or vice versa, and a temperature altering element can be replaced by or interchanged with, a fertility altering element. In other embodiments, both a temperature altering element and fertility altering element can be used.

In certain embodiment, more than one type of element (temperature altering, heating, cooling, fertility altering) can be positioned in the scrotal cushion body.

In one embodiment, the scrotal cushion can comprise a cooling element and a receptacle 420 for receiving the cooling element in the scrotal cushion body.

In another embodiment, the scrotal cushion can comprise a heating element instead of a cooling element, and a receptacle 420 for receiving the heating element in the scrotal cushion body.

In another embodiment, the temperature altering element can provide both cooling and heating.

In one embodiment, the temperature altering cooling or heating element is integral to the scrotal cushion and not removable.

The cooling element can be, for example, selected from the group consisting of a cool pack, an insulated cool pack, a refrigeration device (e.g., a vapor-compression or absorption refrigeration device), and a thermoelectric cooling (Peltier) device. In a specific embodiment, an active cooling element such as a Cold Rush® Cold Therapy System (Össur Americas, 27051 Towne Centre Drive, Foothill Ranch, Calif. 92610 US) is used. In a preferred embodiment, the cooling element is an insulated cool pack. In another embodiment the user can substitute any one temperature altering element for another to fit within the scrotal cushion.

The heating element can be, for example, selected from the group consisting of a heat pack, a heating pad, an insulated heat pack, a thermoelectric heating device or any other heat source.

The receptacle for receiving the cooling or heating element can be, for example, a compartment, cavity or slot of suitable dimensions for receiving the element.

In another embodiment, the receptacle for the cooling or heating element can comprise a removable cover, cap or lid 430. In one embodiment, the removable cap can be removed and replaced by an insulated cool pack or other cooling source or cooling element or by a heat pack or heating element.

In one embodiment the cap 430 is constructed of the same material with the same beneficial properties as the base but has a scrotal “divot” (i.e., indentation or impression) to further accommodate and position the scrotum on the cap.

In other embodiments, the removable cap can be frozen or heated itself or replaced by a cooling or heating element.

In one embodiment, the swollen scrotum can be positioned so that it rests on top of the removable cap or on top of an insulated cool pack that is revealed when the cap is removed.

In another embodiment, the removable cap can be removed from the receptacle, a cooling or heating element inserted into the receptacle 420, and the cap replaced or secured over the receptacle. In this embodiment the cap serves as insulation to prevent over-heating or over-cooling of the scrotum, as a method to increase the scrotal elevation, and as a way to protect the thin scrotal skin. The receptacle and cap can be positioned so that the cooling or heating element in the receptacle is secured in the scrotal cushion body. In use, the scrotum is positioned so that it lies on top of the cooling or heating element (FIG. 4), for example, on top of an insulated cool pack in the receptacle, or on top of the cap or lid of the receptacle receiving the cooling element.

In another embodiment the cap 430 functions as an insulating soft receptacle that envelops the temperature altering element 400. The cap/temperature altering element combination 430/400 can be lifted off of the scrotal cushion body to be heated or cooled and then replaced. Alternatively, the cap/temperature altering element combination 430/400 has a power source that keeps it warm/cool. In this embodiment the cap/temperature altering element could be permanently secured to the scrotal cushion body 100 or removable for maintenance, replenishing the power supply and/or cleaning. In this embodiment a tray 420 is not necessarily needed as the temperature altering element is positioned within the cap. The scrotum then rests on top of the cap/temperature altering element combination.

In another embodiment, the temperature altering element 400 can be removed from the receptacle 420 to be refrigerated, heated or chilled and then replaced or reinserted over the receptacle.

In another embodiment, the cover or cap 430 can be quilted or pillow-like.

In another embodiment, the cap can be removed from the top of the scrotal cushion, to reveal a temperature altering element 400 that rests securely in a tray 420.

In another embodiment, a reusable ice pack or cool pack is provided for use in the scrotal cushion that has an insulated cover to provide a thin layer of insulation between the scrotum and the ice itself. The ice pack preferably has dimension to fit snugly into the plastic tray 420 that can be either removable or fixed on top of the scrotal cushion body 100 (under the removable cover 430). The tray keeps the ice pack from falling off the scrotal cushion. The tray also keeps condensation from soaking the scrotal cushion. It can be removed and emptied in case there is significant amount of condensed water collected in the tray. The scrotum is laid on top of the cap 430 if scrotal elevation is desired without altering the scrotal temperature or conversely, the scrotum is laid on top of the insulated temperature altering element. The insulating sleeve that surrounds the temperature altering element can have all or some of the same beneficial properties as the body, cap and lateral wing elements.

In one embodiment, the scrotal cushion comprises of a body 100 and temperature altering unit 400 and aeration ports 270 and tray 420 and tubes 520 but is without wings 300 or cap 430.

In one embodiment, the scrotal cushion comprises of a body 100 with a cap 430 and temperature altering unit 400 and aeration ports 270 and tubes 520 but is without wings 300.

In one embodiment, the scrotal cushion can be used to position the scrotum but not alter the temperature of the scrotum, e.g., on the operating table for a surgical procedure. Such an embodiment can lack the cover 430, the temperature altering element such as a cooling or heating element 400, and/or aeration ports 270 or aeration tubes 520. In one embodiment, the scrotal cushion is a one-piece molded foam unit for elevating, drying and protecting the scrotum comprising a device body and wings as shown in FIG. 1, but lacking the temperature altering element, the cover and receptacle for the temperature altering element, the aeration elements.

Other fertility altering elements known in the art can be employed. In a specific embodiment, a fertility altering element replaces, or is used in addition to, the temperature altering element. The fertility altering element can use magnetic energy, radio waves, electricity or any other another method of energy exchange known in the art to alter the patient's semen parameters, and thus his fertility, without having to cut or tear open the scrotal skin and underlying fascia. Thus in a specific embodiment, the scrotal cushion can comprise a fertility altering element, which can be positioned in the receptacle 420, instead, or in addition to a temperature altering.

### 5.5. Fabrication of the Scrotal Cushion

The scrotal cushion is preferably constructed of super-absorbent foam that wicks moisture out of the groin and comprises aeration elements that allow for comfortable drying of the perineum. It can be impregnated with antibacterial/antifungal agents such as silver for added benefit with patients at high risk for infections such as Fournier's Gangrene. The tray 420 can be either removable or fixed to the scrotal cushion body with an adhesive. The scrotal cushion is durable enough to be washed and reused or in one embodiment is disposable and designed for approximately 24 hours of use. The scrotal cushion can comprise polymer resins, thermoplastic, thermoset plastic such as temperature-activated thermosetting polymers, phenolic, amino and alkyd resins, diallyl phthalate (DAP), unsaturated polyesters (UP), epoxy resins (EP), vulcanizable rubber, or foam. In a preferred embodiment, the scrotal cushion can be constructed of a single piece of flexible absorbent, hypoallergenic, antibacterial, antifungal, washable, anti-decubitus wicking foam. One foam product that meets most of the above product specifications is SORBATEX™ (Össur Inc., Össur Americas 27051 Towne Centre Drive Foothill Ranch, Calif. 92610 US). In other embodiments, the scrotal cushion body can be constructed of a single piece of soft absorbent foam, except for the tray 420 and the aeration element 270 and/or aeration tube 520, which are preferably constructed of a rigid material such as plastic, which is impermeable to water. Methods for molding foam and plastic are well known in the art, and can include, but are not limited to injection molding, extrusion, blow molding, thermoforming, rotational molding, casting (e.g., mold casting, slush casting, static powder casting), foaming (e.g., mechanical foaming, chemical foaming, physical foaming), structural foam molding, compression molding, transfer molding. In one embodiment, the scrotal cushion is produced by injection molding of polymer resin.

In another embodiment, the scrotal cushion is produced by structural foam molding, which is a category of injection molding. Structural foam molding is a low pressure method of processing thermoplastics, with the most commonly used resin being HDPE (high density polyethylene), although many suitable resins are known in the art, such as acrylonitrile butadiene styrene (ABS), polypropylene (PP) and polyethylene (PE). The end product of structural foam molding is typically a rigid part with a supportive surface, which is suitable for use in the scrotal cushion provided the surface touching the skin has cushioned or anti-decubitus properties.

In a preferred embodiment, the scrotal cushion or portions thereof can be produced by a molded foam process that uses SORBATEX™ foam. Such processes are well known in the art. In a specific embodiment, the body of the scrotal cushion that is contiguous with the lateral wings and/or a separate cap is produced by a molded foam process that uses SORBATEX™ foam. SORBATEX™ foam is well known in the art as a type of medical grade foam padding. It is washable, wicks moisture away from the skin, has high thermal conductivity to keep the skin cool, has antidecubitus properties, it dries quickly and has anti-bacterial properties. This medical grade foam has a history of safe effective medical use, for example, in the manufacturing of cervical spine collars.

### 5.6. Therapeutic Methods Employing the Scrotal Cushion

The scrotal cushion can be employed in a variety of therapeutic methods. Accordingly, in one embodiment, a method is provided for altering the temperature of the scrotum of an individual comprising the steps of:

providing a scrotal cushion as disclosed herein, wherein a temperature altering element is actively cooling or heating or wherein the receptacle for receiving the temperature altering element contains a temperature altering element that is actively cooling or heating;

elevating the individual's scrotum on the scrotal cushion, thereby positioning the scrotum to receive cooling or heating from the temperature altering element;

inserting the first lateral wing element into the groin crease on a corresponding first side of the individual's body and under the leg on that side, thereby compressing the first lateral wing element;

inserting the second lateral wing element into the groin crease on the corresponding second side of the individual's body and under the leg on that side, thereby compressing the second wing element; and

securing the scrotal cushion under the scrotum,

thereby altering the temperature of the scrotum.

In another embodiment, a method is provided for promoting or inhibiting fertility in an individual comprising the steps of:

providing a scrotal cushion as disclosed herein, wherein the temperature altering element is actively cooling or wherein the receptacle for receiving the temperature altering element contains a temperature altering element that is actively cooling;

elevating the individual's scrotum on the scrotal cushion, thereby positioning the scrotum to receive cooling from the temperature altering element;

inserting the first lateral wing element into the groin crease on a corresponding first side of the individual's body and under the leg on that side, thereby compressing the first lateral wing element;

inserting the second lateral wing element into the groin crease on the corresponding second side of the individual's body and under the leg on that side, thereby compressing the second wing element; and

securing the scrotal cushion under the scrotum,

thereby lowering the temperature of the scrotum.

In another embodiment, a method is provided for promoting decreased sperm count or spermatogenesis in an individual comprising the steps of:

providing a scrotal cushion as disclosed herein, wherein the temperature altering element is actively heating or the receptacle for receiving the temperature altering element contains a temperature altering element that is actively heating;

elevating the individual's scrotum on the scrotal cushion, thereby positioning the scrotum to receive heating from the temperature altering element;

inserting the first lateral wing element into the groin crease on a corresponding first side of the individual's body and under the leg on that side, thereby compressing the first lateral wing element;

inserting the second lateral wing element into the groin crease on the corresponding second side of the individual's body and under the leg on that side, thereby compressing the second wing element; and

securing the scrotal cushion under the scrotum,

thereby raising the temperature of the scrotum.

In another embodiment, a method is provided for positioning the scrotum comprising the steps of:

providing a scrotal cushion as disclosed herein;

elevating the individual's scrotum on the scrotal cushion, thereby positioning the scrotum on the scrotal cushion;

inserting the first lateral wing element into the groin crease on a corresponding first side of the individual's body and under the leg on that side, thereby compressing the first lateral wing element;

inserting the second lateral wing element into the groin crease on the corresponding second side of the individual's body and under the leg on that side, thereby compressing the second wing element; and

securing the scrotal cushion under the scrotum.

The method decreases the adjustments needed to secure the scrotal cushion in place in a supine or seated patient.

In another embodiment, a method for altering fertility or semen parameters in an individual is provided. The fertility or semen parameter can be measured, for example, by semen analysis. The method for altering fertility or semen parameters can comprise the steps of:

providing a scrotal cushion as disclosed herein, wherein the temperature altering element is actively cooling or heating or wherein the receptacle for receiving the temperature altering element contains a temperature altering element that is actively cooling or heating;

elevating the individual's scrotum on the scrotal cushion, thereby positioning the scrotum to receive cooling or heating from the temperature altering element;

inserting wing elements of the scrotal cushion into groin creases on each side of the individual's body and under the leg on that side, thereby compressing the wing element; and

securing the scrotal cushion under the scrotum,

thereby altering the temperature of the scrotum and increasing or decreasing fertility.

### 5.7. Therapeutic Uses of the Scrotal Cushion

Scrotal pathology is rarely discussed amongst non-urologists but it is shockingly common and there is little technology available to improve care of the swollen tender scrotum. A method of cooling the scrotum is provided comprising employing the scrotal cushion disclosed herein. The scrotal cushion will decrease pain, reduce swelling of the scrotum, aid in the healing of the swollen scrotum, decrease skin tears and infections that are caused by the current technology and alter fertility. It is equally effective when the patient is sitting or supine. It is easy to insert and may be used either in a health care institution or at home. In one embodiment, the method is used to cool the swollen tender scrotum post-operatively, as described herein.

Scrotal pain/swelling after scrotal surgery is perhaps the most obvious indication for usage of the scrotal wound care device. Commonly performed scrotal surgeries in which the scrotal cushion can be used post-operatively can include, but are not limited to, vasectomy, vasectomy reversal, hydrocelectomy, spermatocelectomy varicocelectomy, penile prosthetics, testicular prosthetics, artificial urinary sphincters, orchidopexy, orchiectomy, scrotal exploration for trauma or suspected torsion. A common surgical complication after scrotal surgery is a hematoma (a collection of clotted blood). This is most often managed with scrotal elevation and ice. The scrotal cushion disclosed herein is ideal for the postoperative management of such complications and is an improvement over the previously described towel/ice technique. With so many scrotal surgeries being performed around the world every day there is a vast demand for a device that improves comfort and wound care for the postoperative tender swollen scrotum.

There are also many nonsurgical causes of the swollen scrotum that would be indications for using the scrotal cushion provided herein. The most common causes of severe scrotal edema in the hospitalized setting are massive resuscitations and anasarca (generalized massive edema) from end stage liver disease or congestive heart failure. In these settings, a melon-sized scrotum is not uncommon. Scrotal elevation is commonly achieved by placing a rolled up towel under the scrotum with or without an ice pack or warm pack. When stretched to this degree, however, the scrotal skin is very thin and weeps moisture from its surface. It is very prone to tearing and abrasions from the towels are common in this setting. The soft elevated surface of the scrotal cushion forms a luxurious protective pillow for the scrotum, decreasing iatrogenia (complications caused by the current art) and increasing patient comfort.

Many embodiments of the scrotal cushion can be produced as described herein with customized, (e.g., ergonomic or attractive) designs.

The difficulty in keeping towels and cool packs centered directly under the scrotum is remedied by the scrotal cushion. In one embodiment, the tray keeps the temperature altering element secured directly under the scrotum thus decreasing the frequent readjusting needed with the towel/ice technique. This feature is designed with disabled patients in mind who may have limited upper extremity dexterity. It is particularly useful for patients with movement disorders, those with upper extremity limitations and for amputees who depend on others for their basic wound care.

Patients with open scrotal, abdominal and/or perineal wounds with persistent drainage would likely benefit from frequent replacement or washing of the scrotal cushion to assist with hygiene and to decrease the risk of opportunistic soft tissue infections.

In another embodiment, a method of altering male fertility is provided comprising employing the scrotal cushion disclosed herein. Cooling and heating of the scrotum have been shown to increase and decrease a man's fertility respectively. In one embodiment, the method comprises employing the scrotal cushion comprising a scrotal cooling element to improve semen parameters and fertility. Scrotal cooling as a treatment for infertility is well described in the medical literature (Laven, 1988; Mulcahy, 1984; Jung, 2001; Schuppe, 2006; Zorgniotti, 1988).

Conversely, in another embodiment the method comprises employing the scrotal cushion comprising a temperature altering element used to warm the scrotum to levels just above normal physiologic temperatures in order to decrease fertility in a patient. Warming the scrotum even a few degrees while sleeping is known to alter semen parameters enough to render a man temporarily infertile. Scrotal/testicular hyperthermia as a form of male contraception is well described in the medical literature, for example, in Robinson D, 1968; Dada, Gupta, & Kucheria, 2003; Kandeel, 1988; Setchell, 1998; and Steinberger, 1991. There are many potential side effects of vasectomy and there are drawbacks to all contraceptive techniques, thus there is great potential for a reversible reliable alternative method of male contraception.

In other embodiments, the scrotal cushion can be used as a cooling device to treat male factor infertility that is caused by varicoceles. A varicocele negatively affects sperm parameters by elevating testicular temperatures that are otherwise autoregulated by the human body. Much of male factor infertility is attributed to varicoceles. In men with infertility associated with a varicocele, scrotal cooling improves semen parameters (Zorgniotti, 1982). This non-surgical infertility treatment could save the health care system thousands of dollars per patient treated.

Elevating the scrotum with the scrotal cushion would be beneficial in scenarios that call for either cooling or warming of the scrotum because the elevation effectively separates the scrotum from surrounding tissues. This allows the temperature altering element to focus its therapeutic effects on the scrotum instead of on the surrounding tissues.

Settings in which the scrotal cushion can be used include, but are not limited to: intensive care unit, vasectomy/urology clinic, infertility clinic, male contraception counseling, surgical ward, medical ward, burn unit, general medical clinic, skilled nursing facility, home use and a military treatment facility.

Nurses and caregivers can employ the scrotal cushion to improve the care of men with swollen tender scrota and to make their work more efficient.

The scrotal cushion offers a number of advantages over current devices known in the art for scrotal treatment. It has a removable cap, a tray under the cap in which a temperature altering element can be secured, a cap that can be cooled/heated, and at least one (and in certain embodiment, a plurality) of active or passive aeration elements. The anti-decubitus nature of the scrotal cushion protects the thin scrotal and perineal skin. Impregnating the scrotal cushion with bactericidal/static and/or antifungal substances provides further protection. Wings serve to wick moisture out of the skin creases and to secure the scrotal cushion in place. The super absorbent construction also serves to wick moisture away from the perineum and out of the surrounding skin crevices.

The scrotal cushion can be scaled in different sizes and/or adapted to be configured for use in adult, bariatric and pediatric patients. Such scaling can be easily calculated and accomplished by one of ordinary skill in the art.

The scrotal cushion can also be scaled in different sizes and/or adapted to be configured for veterinary uses, such as scrotal surgery, involving a male mammal, e.g., cat, dog, cow, horse, pig, sheep, goat, exotic (zoo) mammal, etc.

### 5.8. Index of Numbered Elements

100 Scrotal cushion body (also referred to herein as device body)
110 Proximal-distal axis
120 Lateral axis
210 Proximal side
220 Distal side
230 Opposite (lateral) side
240 Top side
260 Bottom side
270 Aeration element or port
300 Lateral wing element
310 Upper wing portion
320 Lower wing portion
400 Temperature altering (cooling or heating) element or fertility altering element
420 Receptacle or tray for temperature altering element
430 Cap, cover or lid
520 Aeration tube embodiment

The following examples are offered by way of illustration and not by way of limitation.

### 6. EXAMPLES

### 6.1 Example 1

### Use of Scrotal Cushion in Treatment of Polytrauma

A soldier steps on an improvised explosive device and sustains multiple abdominal wounds, a ruptured testicle and amputation of his left leg. He is bed-bound in the intensive care unit undergoing multiple trips to the operating room for life saving procedures. One testicle remains intact but his scrotum is swollen to the size of a large grapefruit and his various other wounds constantly ooze blood and serous fluid down towards his perineum. The scrotal cushion with a scrotal cooling element is used 24 hours per day throughout his 30 day intensive care unit stay providing improved comfort, pain control and hygiene for the patient. The integrity of his remaining scrotal skin is preserved due, in part, to the beneficial properties of the scrotal cushion.

### 6.2 Example 2

### Use of Scrotal Cushion in Treatment After Scrotal Surgery

This example demonstrates the use of the scrotal cushion described herein in the treatment of swelling after scrotal surgery. A male patient presents with scrotal tenderness and swelling after a vasectomy. The patient is prescribed a course of treatment using the scrotal cushion comprising a cooling element for 7 days for 12 hours per day. His symptoms resolve.

### 6.3 Example 3

### Use of Scrotal Cushion in Treatment of Male Factor Infertility

A male patient is evaluated for infertility and is determined to have male factor infertility with normal genetic testing and a varicocele that is causing abnormal sperm motility, morphology and count. The sperm maturation process takes 72 days (Carbone, 1999), thus, instead of surgical removal of his varicocele the patient is prescribed a course of scrotal elevation and cooling for 72 days for 8 hours every night. If the semen analysis parameters improve the physician recommends continued use of the scrotal cushion along with unprotected intercourse until a child is conceived. If semen parameters do not improve the patient is offered either another 72 day treatment period or surgery.

### 6.4 Example 4

### Use of Scrotal Cushion as Reversible Contraceptive

A man seeking a contraceptive technique is prescribed a course of treatment using the scrotal cushion comprising the step of warming the scrotum for 72 days for 8 hours every night.

After the treatment period and before having unprotected intercourse, a semen analysis is performed. If the semen parameters are sufficiently decreased, knowing that no contraceptive method is 100% effective, the patient can proceed with unprotected intercourse.

### REFERENCES CITED

Carbone. (1999). Male Reproductive physiology and assisted reproductive technology, AUA Update Series, Vol 18, Lesson 21.
Dada, Gupta, & Kucheria. (2003). Spermatogenic arrest in men with testicular hyperthermia. Teratogenesis, Carcinogenesis, and Mutagenesis, S1 235-43.
Jung. (2001) Improvement of semen quality by nocturnal scrotal cooling and moderate behavioral change to reduce genital heat stress in men with with oligoasthenoteratozoospermia. Reproduction, 121, 595-603.
Kandeel, F. (1988). Role of temperature in regulation of spermatogenesis and the use of heating as a method for contraception. Fertility and Sterility, 49(1): 1-23.
Laven, J. (1988). Influence of occupation and living habits on semen quality in men (scrotal insulation and semen quality). European Journal of Obstetrics & Gynecology & Reproductive Biolog, 137-141.
Mulcahy. (1984). Scrotal hypothermia and the infertile man”. J Urol, 132:469-70.
Robinson D, R. J. (1968). Control of human spermatogenesis by induced changes of intrascrotal temperature. JAMA, 204(4):80-87.
Schuppe. (2006). Improvement of semen quality by nocturnal scrotal cooling in oligospermic men with a history of testicular maldescent. International journal of andrology, 28(2):93-8.
Setchell, B. (1998). The Parkes lecture: Heat and the testes. Fertility and Sterility, (pp. 114(2): 179-94).
Steinberger, A. (1991). Effects of temperature on the biochemistry of the testis. Advances in Experimental Medicine and Biology, 286:33-47.
Zorgniotti. (1982). Further clinical experience with testis hypothermia for infertility due to poor semen. Urology, June; 19(6):636-40.
Zorgniotti. (1988). Measurement of intrascrotal temperature in normal and subfertile men. J. Reprod. Fert., 82, 563-566.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

What is claimed is:

1. A scrotal cushion, comprising:
a scrotal cushion body, wherein the scrotal cushion body has a longitudinal (proximal-distal) axis and a lateral axis, and wherein the scrotal cushion body comprises:
a proximal side and a distal side,
first and second opposite sides, wherein the first and second opposite sides are positioned opposite with respect to the lateral axis,
a top side,
a bottom side opposite the top side,
at least one soft, absorbent material for providing a supporting substrate; and
first and second lateral wing elements secured to each of the first and second sides respectively of the scrotal cushion body.

2. The scrotal cushion of claim 1 comprising a temperature altering element or a fertility altering element.

3. The scrotal cushion of claim 2, wherein the temperature altering element is an insulated heating or a cooling element.

4. The scrotal cushion of claim 2 wherein the temperature altering element is selected from the group consisting of a cool pack, a refrigeration device, a vapor-compression device, an absorption refrigeration device, a thermoelectric cooling device, a heat pack, a heating pad, and a thermoelectric heating device.

5. The scrotal cushion of claim 1 comprising a receptacle for receiving a temperature altering element or a fertility altering element.

6. The scrotal cushion of claim 5 wherein the receptacle for receiving the temperature altering element or fertility altering element comprises a removable cap or lid.

7. The scrotal cushion of claim 6 wherein the removable cap or lid is positioned on the top side, the bottom side, the proximal side, the distal side or the first or second opposite side.

8. The scrotal cushion of claim 1, wherein the first and second lateral wing elements are secured on each of the first and second sides.

9. The scrotal cushion of claim 1, wherein the first or the second lateral wing element comprises an upper wing portion and a lower wing portion.

10. The scrotal cushion of claim 1 comprising at least one aeration element positioned in the scrotal cushion body.

11. The scrotal cushion of claim 10 wherein the at least one aeration element is a port or a tube.

12. The scrotal cushion of claim 11 wherein a second aeration element is positioned within the port or tube.

13. A method for altering the temperature of the scrotum of an individual comprising the steps of:
providing the scrotal cushion of claim 2, wherein the temperature altering element is actively cooling or heating or the fertility altering element is active, or
providing the scrotal cushion of claim 5, wherein the receptacle for receiving the temperature altering element contains a temperature altering element that is actively cooling or heating or a fertility altering element that is active:
elevating the individual's scrotum on the scrotal cushion, thereby positioning the scrotum to receive cooling or heating from the temperature altering element or activity from the fertility altering element;
inserting the first lateral wing element into the groin crease on a corresponding first side of the individual's body and under the leg on that side, thereby compressing the first lateral wing element;
inserting the second lateral wing element into the groin crease on the corresponding second side of the individual's body and under the leg on that side, thereby compressing the second wing element; and
securing the scrotal cushion under the scrotum,
thereby altering the temperature of the scrotum and/or altering the fertility of the individual.

14. A method for inhibiting or promoting fertility in an individual comprising the steps of:
providing the scrotal cushion of claim 2, wherein the temperature altering element is actively cooling or heating or the fertility altering element is active to inhibit or promote fertility, or
providing the scrotal cushion of claim 5, wherein the receptacle for receiving the temperature altering element contains a temperature altering element that is actively cooling or heating or a fertility altering element that is active to inhibit or promote fertility;
elevating the individual's scrotum on the scrotal cushion, thereby positioning the scrotum to receive cooling or heating from the temperature altering element or activity from the fertility altering element;
inserting the first lateral wing element into the groin crease on a corresponding first side of the individual's body and under the leg on that side, thereby compressing the first lateral wing element;
inserting the second lateral wing element into the groin crease on the corresponding second side of the individual's body and under the leg on that side, thereby compressing the second wing element; and
securing the scrotal cushion under the scrotum,
thereby altering the temperature of the scrotum and/or altering the fertility of the individual.

15. A method for promoting decreased sperm count or spermatogenesis in an individual comprising the steps of:
providing the scrotal cushion of claim 2, wherein the temperature altering element is actively heating or the fertility altering element is active to decrease sperm count, or providing the scrotal cushion of claim 5, wherein the receptacle for receiving the temperature altering element contains a temperature altering element that is actively heating or a fertility altering element that is active to decrease sperm count;
elevating the individual's scrotum on the scrotal cushion, thereby positioning the scrotum to receive heating from the temperature altering element or activity from the fertility altering element;
inserting the first lateral wing element into the groin crease on a corresponding first side of the individual's body and under the leg on that side, thereby compressing the first lateral wing element;
inserting the second lateral wing element into the groin crease on the corresponding second side of the individual's body and under the leg on that side, thereby compressing the second wing element; and
securing the scrotal cushion under the scrotum,
thereby raising the temperature of the scrotum and/or decreasing the fertility of the individual.

16. A method for promoting increased sperm count or spermatogenesis in an individual comprising the steps of:
providing the scrotal cushion of claim 2, wherein the temperature altering element is actively cooling or the fertility altering element is active to increase sperm count, or providing the scrotal cushion of claim 5, wherein the receptacle for receiving the temperature altering element contains a temperature altering element that is actively cooling or a fertility altering element that is active to increase sperm count;
elevating the individual's scrotum on the scrotal cushion, thereby positioning the scrotum to receive cooling from the temperature altering element or activity from the fertility altering element;
inserting the first lateral wing element into the groin crease on a corresponding first side of the individual's body and under the leg on that side, thereby compressing the first lateral wing element;
inserting the second lateral wing element into the groin crease on the corresponding second side of the individual's body and under the leg on that side, thereby compressing the second wing element; and
securing the scrotal cushion under the scrotum,
thereby lowering the temperature of the scrotum and/or increasing the fertility of the individual.
